(19) United States
(12) Patent Application Publication
(10) Pub. No.: US 2009/0025113 A1
(43) Pub. Date: Jan. 29, 2009

(54) Visor

(75) Inventor: Brian James Kelleghan, Longmont, CO (US)

Correspondence Address: PATENT LAW OFFICES OF RICK MARTIN, PC, PO BOX 1839, LONGMONT, CO, 80502, US

(73) Assignee: BISON DESIGNS, LLC, Longmont, CO (US)

(21) Appl. No.: 12/178,549

(22) Filed: Jul. 23, 2008

Related U.S. Application Data
(60) Provisional application No. 60/954,868, filed on Aug. 9, 2007.
(60) Provisional application No. 60/951,353, filed on Jul. 23, 2007.

(51) Int. Cl. A61F 9/00; A42B 1/00

(52) U.S. Cl. 2/12

(57) ABSTRACT

A visor molded as a single piece that has stretch so it fits more than one size. The visor is made from a stretchable polymer. Also grooves can be provided in the headband to allow for further flexibility. The visor also has molded grooves and stitching lines, to allow the visual concealment of actual stitching lines to attach sweatbands and other features. Also provides are holes for attaching decorative studs.

### CROSS REFERENCE APPLICATIONS

This application is a non-provisional application claiming the benefits of provisional application No. 60/951,353 filed on Jul. 23, 2007 and provisional application No. 60/954,868 filed on Aug. 9, 2007.

### BACKGROUND

Molded plastic visors are well known. Most have several drawbacks. Either the headband has to be formed incomplete, with a space at the back, or the visor has to be made in a number of sizes, to accommodate different head sizes. Also, plastic visors are often uncomfortable to wear in hot weather, because the plastic headband holds heat and sweat against the user's head and because many of them are molded of fairly rigid plastic. Sweatbands have in the past been glued into the inside of the headband, but this reduces the absorption of sweat and can make the fabric stiff or lumpy and therefore less comfortable. If the sweatband is sewn onto the visor, the stitching line creates an obvious visual change to the outside of visor.

The foregoing example of the related art and limitations related therewith are intended to be illustrative and not exclusive. Other limitations of the related art will become apparent to those of skill in the art upon a reading of the specification and a study of the drawings.

### SUMMARY

It is one aspect of the present invention to provide a molded visor that is variable in size and comfortable to wear.

The following embodiments and aspects thereof are described and illustrated in conjunction with systems, tool and methods which are meant to be exemplary and illustrative, not limiting in scope. In various embodiments, one or more of the above described problems have been reduced or eliminated, while other embodiments are directed to other improvements.

The visor is made from flexible foam rubber with stretch built in to the foam. Also the headband can have grooves to provide for airflow and allow extra stretch. Further the visor has molded stitching lines, which allow the actual stitches to blend in with the molded lines. The molded stitching lines have generally oblong holes that at least partially overlapping them, reducing the visual appearance of the holes. These holes allow decorative studs or other devices to be attached to the visor without causing damage to the visor. A hat with a visor can also have a bill with the molded stitching and holes.

In addition to the exemplary aspects and embodiments described above, further aspects and embodiments will become apparent by reference to the accompanying drawings forming a part of this specification wherein like reference characters designate corresponding parts in the several views.

### BRIEF DESCRIPTION OF THE DRAWINGS

FIG. 1 is a top plan view of a visor.

FIG. 2 is a bottom plan view of a visor.

FIG. 3 is a side elevation view of either side of the visor.

FIG. 4 is a front elevation view of the visor.

FIG. 5 is a back plan view of the visor.

FIG. 6 is a top perspective view of the visor.

FIG. 7 is a top plan view of an alternate embodiment of the visor with ventilation channels in the band.

FIG. 8 is a bottom plan view of the visor with channels.

FIG. 9 is a top perspective view of the visor with channels.

FIG. 10 is a top plan view of an alternate embodiment of the visor.

FIG. 11 is a bottom plan view of FIG. 10.

FIG. 12 is a side elevation view of either side of the alternate embodiment of the visor.

FIG. 13 is a front elevation view of the alternate embodiment of the visor.

FIG. 14 is a back plan view of the alternate embodiment of the visor.

FIG. 15 is a top perspective view of the alternate embodiment of the visor.

FIG. 16 is a blown up view of the bill of a visor.

FIG. 17 is a top plan view of a second alternate embodiment of the visor.

FIG. 18 is a bottom plan view of FIG. 17.

FIG. 19 is a right side elevation view of the second alternate embodiment of the visor.

FIG. 20 is a left side elevation view of the second alternate embodiment of the visor.

FIG. 21 is a back plan view of the second alternate embodiment of the visor.

FIG. 22 is a top perspective view of the second alternate embodiment of the visor.

Before explaining the disclosed embodiment of the present invention in detail, it is to be understood that the invention is not limited in its application to the details of the particular arrangement shown, since the invention is capable of other embodiments. Exemplary embodiments are illustrated in referenced figures of the drawings. It is intended that the embodiments and figures disclosed herein are to be considered illustrative rather than limiting. Also, the terminology used herein is for the purpose of description and not of limitation.

### DETAILED DESCRIPTION OF THE DRAWINGS

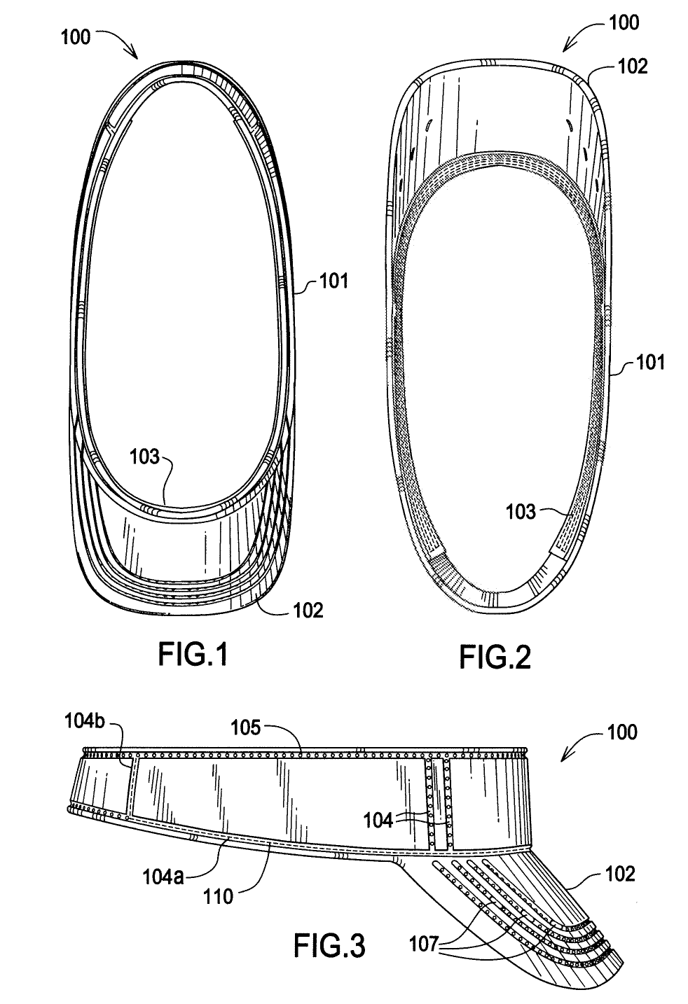

Referring first to FIGS. 1 to 5, the visor 100 has a band 101 and a bill 102. The visor 100 is molded as a single piece from a flexible polymer such as foam rubber. Lighter weight EVA foam rubber to more durable EPR foam rubber can be used depending on the application. Other types of flexible polymer would work as well. Foam rubber is crushable, but has excellent “memory” to return to the original shape with little or no cosmetic damage. It is desirable that the polymer be selected such that the band 101 can stretch to allow for user comfort and a “one size fits most” feature. It is believed that a foam rubber will increase in volume in the range of 5% to 50% from mold to release from mold and curing will work well. A buoyant material would work well for visors designed for water sports use.

The visor 100 can be molded in a range of sizes to allow for children, teens and adults to wear the visor 100. The visor 100 can have a sweatband 103, best seen in FIG. 6, attached inside the band 101. The sweatband 103 is made of absorbent material or wicking material, most often a fabric such as cotton. In the depicted embodiment, the sweatband 103 does not extend all the way round the inside of the band 101. If the sweatband 103 does extend all the way around, the sweat band 103 will need to be made of a stretchable, absorbent material.

The visor 100 has grooves 104 with stitching lines 105 molded into the material. The molded stitching lines 105 in the grooves 104 allow the sweatband 103, and any other desired add on, to be stitched to the visor 100 without creating an obvious impact on the visual appearance of the visor 100 and camouflaging the actual stitching. The groove 104 and molded stitching line 105 is best seen in FIG. 16. The groove 104 means that the actual stitching does not create a groove on the surface of the visor, and prevents puckering of the surface, which is not desirable visually.

The sweatband 103 in the depicted embodiment is sewn down along the bottom edge in groove 104a and up to the top of the headband 101 in grooves 104b on each side 106 of the visor 100. The sweatband 103 can be sewn down along any of the grooves 104. Further the grooves 104 provide a guide for the placement of the stitching. The grooves 104 can be place in a different location than the ones in the depicted embodiment to allow for different arrangements of the sweatband 103.

Molded into the grooves 104 on the bill 102 of the visor 100 are small slits 107, seen in FIGS. 3, 5, 8, 9, 11, and 16. The slits 107 are sized to allow a decorative stud 108, such as a Jibbitz® stud, to be placed on the visor and are formed as roughly oblong holes though the bill of the visor. The stretchable nature of polymer the visor 100 is made from allows the slits 107 to stretch to allow the stud 108 to be passed through the slit, as seen in FIG. 16. The slit 107 placed in the groove 104 is not highly visible when the stud 108 is not in place. This allows for greater flexibility of the visual appearance of the visor 100.

In an alternate embodiment seen in FIGS. 12, 13 and 15 of the visor 200 has a number of holes 202 preformed into the band 201. The band 201 has the groove 104 and molded stitching 105 as in the previous embodiment. These holes 202 allow for the placement of a large number of studs 108.

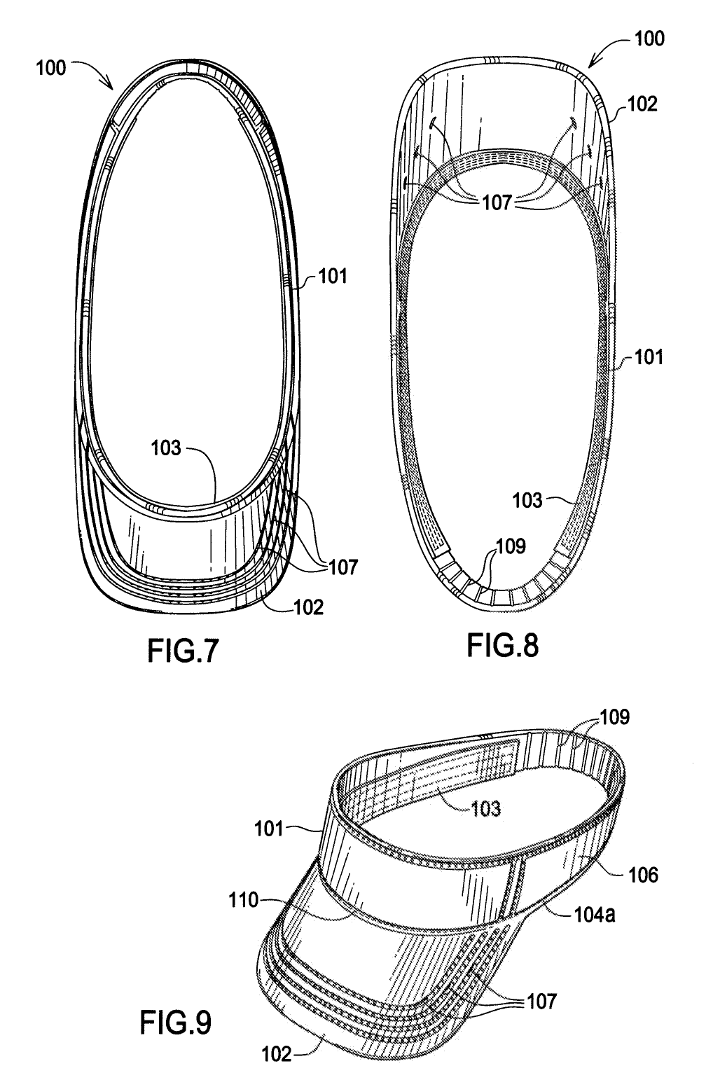

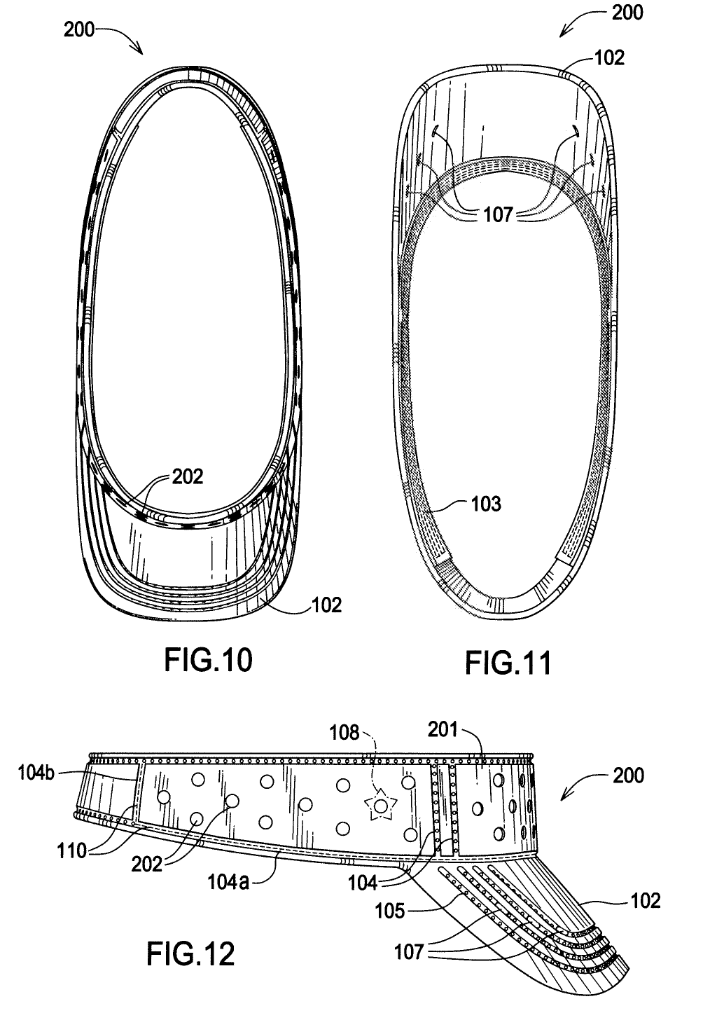

Referring next to FIGS. 7, 8 and 9, the band 101 can have channels 109 running vertically along the inside of the band 101. The channels 109 allow for greater air circulation and create greater flexibility in the band 101. The channels 109 can be located on some or all of the inside surface of the band. The channels 109 can have a depth in the range of about 10% to about 50% of the thickness of the band 101. The depth of the channels 109 will depend on the amount of stretch desired, the polymer chosen and other factors.

In another embodiment (not shown) the visor 100 can be used as a base to add additional fabric, such as to form a hat.

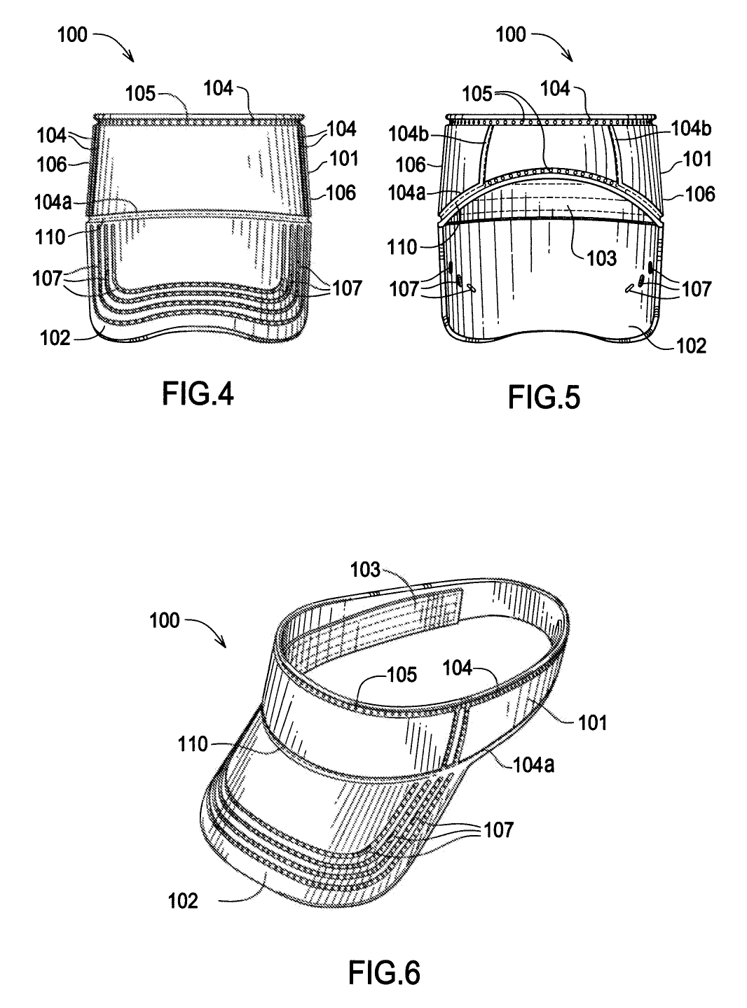

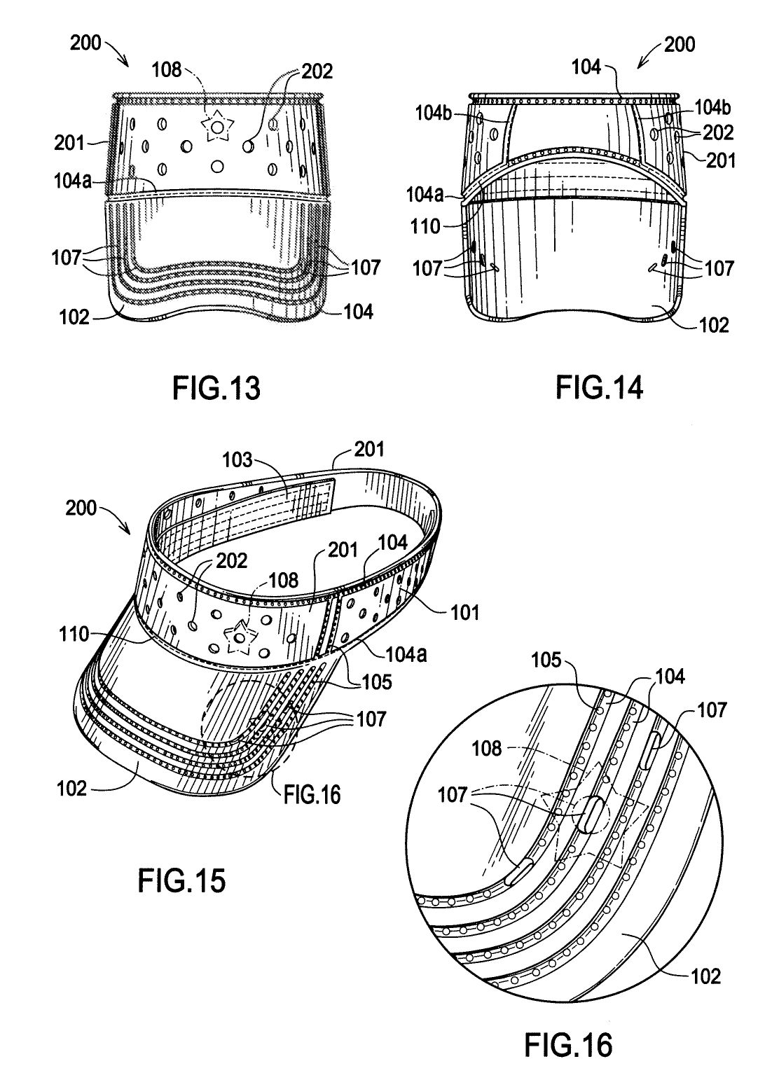

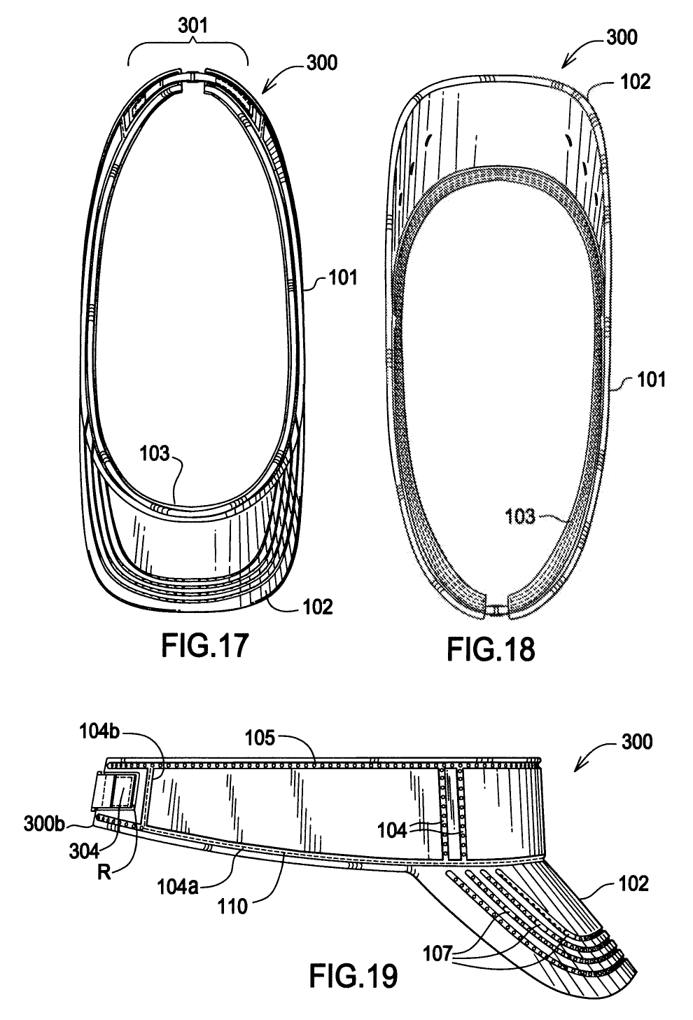

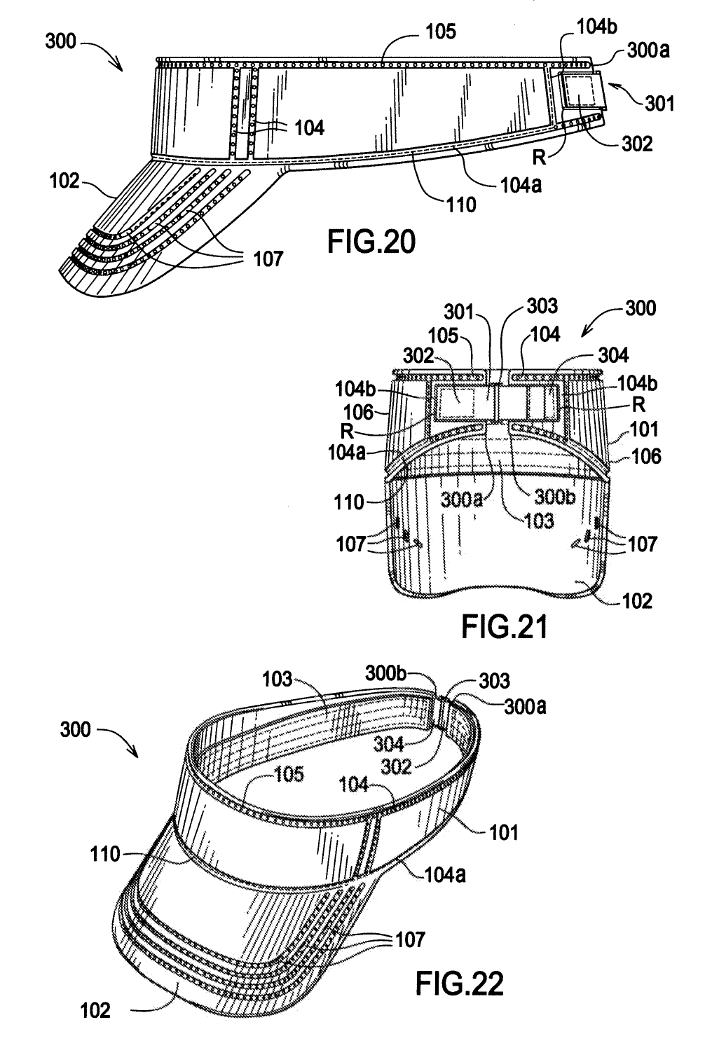

In another embodiment the visor 300 has hook and loop fabric fastener 301 to allow for greater adjustability, as shown in FIGS. 17-22. The front of visor 300 can either be the visor shown in FIG. 4 or in FIG. 13. The visor 300 is split at the back and fabric loop 302 with ring 303 is attached to one side 300a, in the depicted embodiment by sewing. On the other side 300b a strip 304 has hook fabric on one end and loop fabric on the other to allow the strip 304 to attached to itself in a known manner. When strip 304 is fed through ring 303 the faster 301 is closed in a known manner. The adjustment of the amount of strip 304 fed through ring 303 will adjust the size of visor 300. The ends 300a and 300b can have small recesses R formed in them for the strips 302 and 304 to be attached down into them to create a lower visual profile for the fastener 301. Additionally the sweat band 103 can extend all the way to the ends 300a and 300b to mask the stitching of the strips.

While a number of exemplary aspects and embodiments have been discussed above, those of skill in the art will recognize certain modifications, permutations, additions and sub-combinations therefore. It is therefore intended that the following appended claims hereinafter introduced are interpreted to include all such modifications, permutations, additions and sub-combinations are within their true sprit and scope. Each apparatus embodiment described herein has numerous equivalents.

I claim:

1. A piece of headgear comprising:
a headband which substantially encircles a head of a wearer;
a bill attached to the headband;
at least one groove formed into a body of the headgear;
at least one substantially oblong hole located at least partially within the groove; and
said oblong hole extending through the body and capable of being deformed to allow a decorative item to be attached to the headgear.

2. The headgear of claim 1 further comprising a molded stitching line in the groove.

3. The headgear of claim 2, wherein the headgear is a visor.

4. The headgear of claim 2, wherein at least one groove is located on the bill.

5. The headgear of claim 4, wherein the groove is located near a periphery of the bill.

6. The headgear of claim 1, wherein the oblong hole is entirely with the groove.

7. The headgear of claim 1, further comprising a second groove in the headband to allow a sweat band to be sewn to the inside of the headband with the stitching inside the second groove.

8. A method of forming a visor capable of having a decorative stud attached without damaging the visor:
creating a piece of headgear out of a flexible polymer, said headgear having a headband and a bill;
forming a groove into the headgear;
forming a slit through the headgear, said slit at least partially overlapping the groove;
said slit being formed such that it is minimally visible to a viewer.

9. The method of claim 8 further comprising molding a stitching line in the groove.

10. The method of claim 9, wherein the headgear is a visor.

11. The method of claim 9, wherein at least one groove is located on the bill.

12. The method of claim 9, wherein the groove is located near a periphery of the bill.

13. The method of claim 8, wherein the slit is entirely with the groove.

14. The method of claim 8, further comprising molding a second groove in the headband to allow a sweat band to be sewn to the inside of the headband with the stitching inside the second groove.
